(19) United States
(12) Patent Application Publication
(10) Pub. No.: US 2024/0082764 A1
(43) Pub. Date: Mar. 14, 2024

(54) FILTER, GAS SENSOR, AND GAS SENSOR SYSTEM

(71) Applicant: KABUSHIKI KAISHA TOSHIBA, Tokyo (JP)

(72) Inventors: Akira FUJIMOTO, Kawasaki Kanagawa (JP); Yosuke AKIMOTO, Yokohama Kanagawa (JP); Hiroaki YAMAZAKI, Yokohama Kanagawa (JP)

(73) Assignee: KABUSHIKI KAISHA TOSHIBA, Tokyo (JP)

(21) Appl. No.: 18/175,004

(22) Filed: Feb. 27, 2023

(30) Foreign Application Priority Data
Sep. 14, 2022 (JP) 2022-146126

(51) Int. Cl. B01D 39/16; B01D 19/00; B01D 39/20; G01N 33/00

(52) U.S. Cl. CPC B01D 39/1692; B01D 19/0031; B01D 39/2031; G01N 33/0011; B01D 2239/065

(57) ABSTRACT

According to one embodiment, a filter includes a first layer and a second layer. The first layer includes a first resin provided with a plurality of holes. The first layer includes a first face. At least a part of the plurality of holes reaches the first face. The second layer includes a second resin. The second resin blocks at least a part of the plurality of holes reaching the first face.

### CROSS-REFERENCE TO RELATED APPLICATIONS

This application is based upon and claims the benefit of priority from Japanese Patent Application No. 2022-146126, filed on Sep. 14, 2022; the entire contents of which are incorporated herein by reference.

### FIELD

Embodiments described herein generally relate to a filter, a gas sensor, and a gas sensor system.

### BACKGROUND

For example, a filter is used for a gas sensor. Filters are required to have improved characteristics.

### BRIEF DESCRIPTION OF THE DRAWINGS

FIG. 1A and FIG. 1B are schematic cross-sectional views illustrating a filter according to a first embodiment;

FIG. 2 is a schematic cross-sectional view illustrating a filter according to the first embodiment;

FIG. 3A and FIG. 3B are schematic cross-sectional views illustrating a filter according to the first embodiment;

FIG. 4 is a schematic cross-sectional view illustrating a filter according to the first embodiment;

FIG. 5 is a schematic cross-sectional view illustrating a filter according to the first embodiment;

FIG. 6 is an electron micrograph image of the filter according to the first embodiment;

FIG. 7 is a schematic cross-sectional view illustrating a sensor according to a second embodiment; and

FIG. 8 is a schematic cross-sectional view illustrating a sensor according to a third embodiment.

### DETAILED DESCRIPTION

According to one embodiment, a filter includes a first layer and a second layer. The first layer includes a first resin provided with a plurality of holes. The first layer includes a first face. At least a part of the plurality of holes reaches the first face. The second layer includes a second resin. The second resin blocks at least a part of the plurality of holes reaching the first face.

Various embodiments are described below with reference to the accompanying drawings.

The drawings are schematic and conceptual; and the relationships between the thickness and width of portions, the proportions of sizes among portions, etc., are not necessarily the same as the actual values. The dimensions and proportions may be illustrated differently among drawings, even for identical portions.

In the specification and drawings, components similar to those described previously or illustrated in an antecedent drawing are marked with like reference numerals, and a detailed description is omitted as appropriate.

### FIRST EMBODIMENT

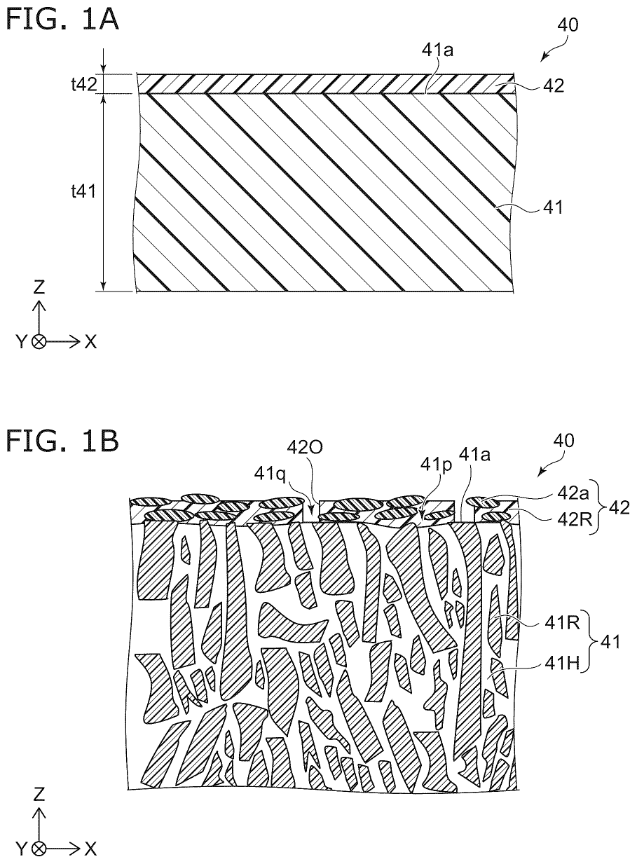

FIG. 1A and FIG. 1B are schematic cross-sectional views illustrating a filter according to the first embodiment.

As shown in FIG. 1A, a filter 40 according to the embodiment includes a first layer 41 and a second layer 42. A direction from the first layer 41 to the second layer 42 is defined a Z-axis direction. One direction perpendicular to the Z-axis direction is defined as an X-axis direction. A direction perpendicular to the Z-axis direction and the X-axis direction is defined as a-Y axis direction. The first layer 41 and the second layer 42 extend along the X-Y plane. The filter 40 is, for example, a film.

As shown in FIG. 1B, the first layer 41 includes a first resin 41R. The first resin 41R is provided with a plurality of holes 41H. The first layer 41 is, for example, a porous layer.

The first layer 41 includes a first face 41a. The first face 41a is a face facing the second layer 42. At least a part of the plurality of holes 41H reaches the first face 41a.

The second layer 42 is provided on the first face 41a of the first layer 41. The second layer 42 includes a second resin 42R. The second resin 42R blocks at least a part of the plurality of holes 41H reaching the first face 41a. The second layer 42 is, for example, an ineffective void layer. A part of the second resin 42R may be provided in a portion near the surface of the hole 41H.

In the embodiment, the first layer 41 being porous and the second resin 42R (the second layer 42) partially blocking the plurality of holes 41H of the first layer 41 are provided. For example, other part of holes 41H in the first layer 41 are not blocked by the second resin 42R.

By the hole 41H which is not blocked by the second resin 42R, a target gas can pass through the filter 40. The target gas is, for example, hydrogen. On the other hand, since a part of the plurality of holes 41H is blocked by the second resin 42R, non-target substance does not pass through the filter 40. Non-target substances include, for example, liquids (water, oil, and the like). According to the embodiment, the target gas can be efficiently permeated. Permeation of non-target substances is suppressed. A filter capable of improving characteristics can be provided.

According to the filter according to the embodiment, for example, high water repellency can be obtained. For example, high breathability can be obtained. For example, high chemical resistance can be obtained. For example, high corrosion resistance can be obtained. For example, high dust resistance can be obtained. For example, entry of water or oil can be suppressed. For example, high reliability can be obtained.

In the embodiment, the first resin 41R preferably includes a fluorine compound. The first resin 41R includes, for example, PTFE (polytetrafluoroethylene). Stable permeability can be obtained. The permeation of water, etc. can be effectively suppressed.

The second resin 42R includes, for example, an acrylic resin. Thereby, at least a part of the plurality of holes 41H is stably blocked. For example, permeation of non-target substance can be stably suppressed.

A thickness of the first layer 41 is defined as a first thickness t41. A thickness of the second layer 42 is defined as a second thickness t42. These thicknesses are lengths along the Z-axis direction. In the embodiment, for example, the first thickness t41 is thicker than the second thickness t42. In one example, the first thickness t41 is 2 times or more the second thickness t42. For example, the first thickness t41 may be 10,000 times or less of the second thickness t42. For example, the permeability of the target gas and the suppression of the permeation of non-target substance can be appropriately obtained.

The first thickness t41 of the first layer 41 is, for example, not less than 10 μm and not more than 5000 μm. The first thickness t41 may be, for example, 1000 μm or less. The second thickness t42 of the second layer 42 is, for example, not less than 0.1 μm and not more than 100 μm. In a case where a part of the second resin 42R is provided in a part of the hole 41H near the surface, the thickness of the second resin 42R provided in the part of the hole 41H near the surface may be, for example, not less than 0.1 μm and not more than 5 μm.

The second resin 42R covers at least a part of the first face 41a. As shown in FIG. 1B, the first face 41a includes a first region 41p in which the second resin 42R is provided and a second region 41q in which the second resin 42R is not provided. The ratio of the area of the first region 41p to the area of the second region 41q is not less than 0.01 and not more than 100.

For example, the second resin 42R may include an opening 420. The second region 41q where the second resin 42R is not provided corresponds to the opening 420. The opening ratio may be, for example, not less than 1% and not more than 99%.

As shown in FIG. 1B, in this example, the second layer 42 further includes a plurality of first solid pieces 42a. The plurality of first solid pieces 42a are fixed by the second resin 42R. The plurality of first solid pieces 42a include, for example, at least one of a metal, a metal oxide or a metal nitride. For example, the plurality of first solid pieces 42a include Fe, Cr and Ni. The plurality of first solid pieces 42a include, for example, SUS (Steel Use Stainless). For example, high corrosivity is obtained. The plurality of first solid pieces 42a may include, for example, titanium oxide.

The average size (length) of one of the plurality of first solid pieces 42a is, for example, not less than 0.1 μm and not more than 10 μm.

In a first sample and a second sample related to the filter according to the embodiment, the plurality of first solid pieces 42a are provided. In the first sample and the second sample, the plurality of first solid pieces 42a include Fe, Cr and Ni. In the first sample, the second thickness t42 of the second layer 42 is 20 μm. In the second sample, the second thickness t42 of the second layer 42 is 40 μm.

In a third sample, the first layer 41 is provided and the second layer 42 is not provided. In the first to third samples, the first resin 41R of the first layer 41 is PTFE.

The filters of these samples are provided in the opening of the housing. The state of the environment inside the housing is evaluated when the environment outside the housing (out space) is changed.

In the first sample, in a case where the out space humidity is 90% RH, the internal humidity is 65% RH. In the first sample, in a case where the out space includes hydrogen at a concentration of 1% (volume %), the concentration of hydrogen in the internal space is 0.6%.

In the second sample, in the case where the out space humidity is 90% RH, the internal humidity is 55% RH. In the first sample, in the case where the out space includes hydrogen at a concentration of 1% (volume %), the concentration of hydrogen in the internal space is 0.3%.

In the third sample, in the case where the out space humidity is 90% RH, the internal humidity is 85% RH. In the first sample, in the case where the out space includes hydrogen at a concentration of 1% (volume %), the concentration of hydrogen in the internal space is 0.8%.

As described above, in the first sample and the second sample, by providing the filter 40 according to the embodiment, the entry of water into the housing can be effectively suppressed. Hydrogen can permeate the first sample and the second sample. Decrease in the permeability of hydrogen by the filter 40 according to the embodiment is relatively small. In the embodiment, high permeability for the target gas (hydrogen) and low permeability for the non-target substance (water) are obtained.

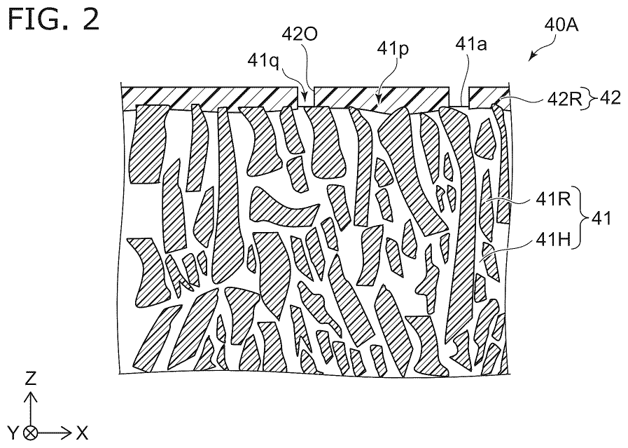

FIG. 2 is a schematic cross-sectional view illustrating a filter according to the first embodiment.

As shown in FIG. 2, a filter 40A according to the embodiment includes the first layer 41 and the second layer 42. In the filter 40A, the plurality of first solid pieces 42a are omitted. The configuration of the filter 40A except for this may be the same as the configuration of the filter 40. The filter 40A also provides a high permeability for the target gas and a low permeability for the non-target substance.

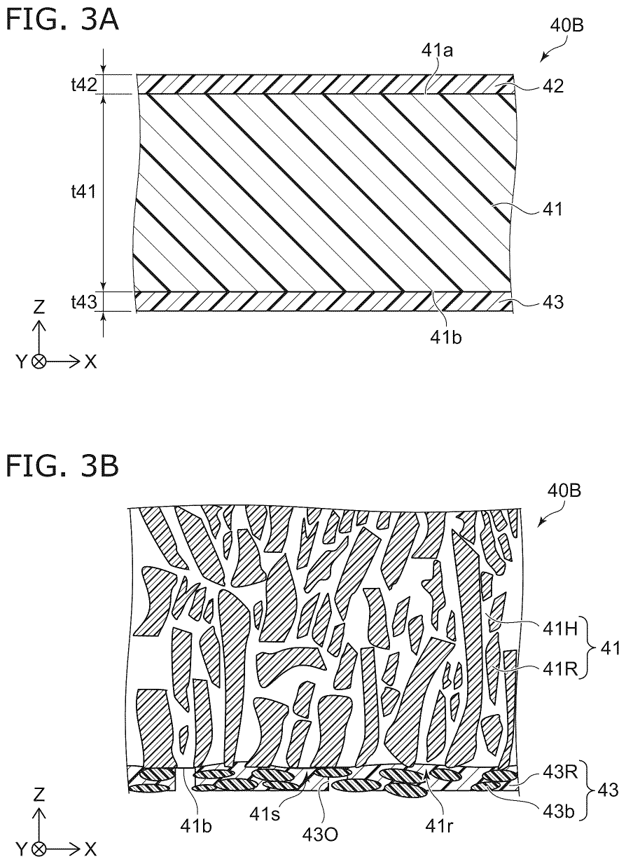

FIG. 3A and FIG. 3B are schematic cross-sectional views illustrating a filter according to the first embodiment.

As shown in FIG. 3A, a filter 40B according to the embodiment includes the first layer 41, the second layer 42, and a third layer 43. The configuration of the filter 40B other than this may be the same as the configuration of the filter 40 or the filter 40A.

As shown in FIG. 3A, the first layer 41 is located between the third layer 43 and the second layer 42. The first layer 41 includes a second face 41b facing the third layer 43.

As shown in FIG. 3B, the third layer 43 includes a third resin 43R. A part of the plurality of holes 41H of the first layer 41 reaches the second face 41b. The third resin 43R partially blocks the plurality of holes 41H reaching the second face 41b. For example, the third layer 43 includes an opening 430.

For example, the third resin 43R includes an acrylic resin. The third thickness t43 of the third layer 43 is, for example, not less than 0.1 μm and not more than 100 μm.

The third resin 43R covers at least a part of the second face 41b. For example, the second face 41b includes a third region 41r in which the third resin 43R is provided and a fourth region 41s in which the third resin 43R is not provided. The ratio of the area of the third region 41r to the area of the fourth region 41s is, for example, not less than 0.01 and not more than 100.

As shown in FIG. 3B, the third layer 43 may further include a plurality of second solid pieces 43b. By providing the plurality of second solid pieces 43b, for example, higher corrosion resistance can easily be obtained. The material of the plurality of second solid pieces 43b may be the same as the material of the plurality of first solid pieces 42a. The material of the plurality of second solid pieces 43b may be different from the material of the plurality of first solid pieces 42a.

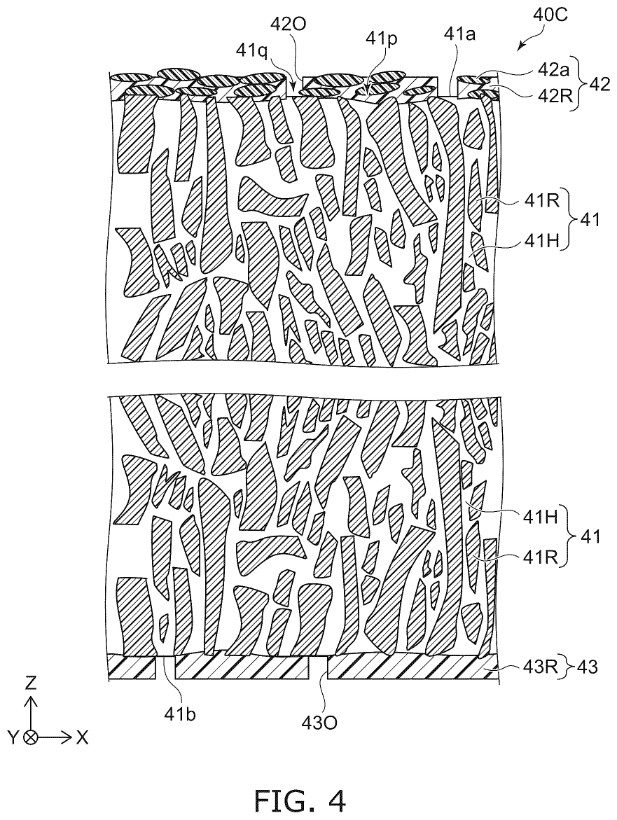

FIG. 4 is a schematic cross-sectional view illustrating a filter according to the first embodiment.

As shown in FIG. 4, a filter 40C according to the embodiment includes the first layer 41, the second layer 42, and the third layer 43. In the filter 40C, the second layer 42 includes the plurality of first solid pieces 42a. In the third layer 43, the plurality of second solid pieces 43b are omitted. The configuration of the filter 40C except for this may be the same as the configuration of the filter 40B. The filter 40C also provides a high permeability for the target gas and a low permeability for the non-target substance.

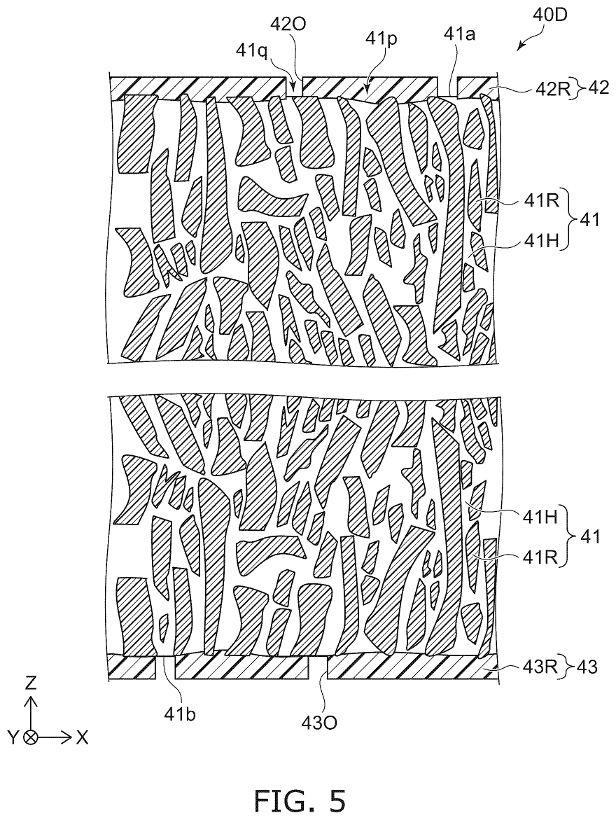

FIG. 5 is a schematic cross-sectional view illustrating a filter according to the first embodiment.

As shown in FIG. 5, a filter 40D according to the embodiment includes the first layer 41, the second layer 42, and the third layer 43. In the filter 40D, the plurality of first solid pieces 42a are omitted in the second layer 42. In the third layer 43, the plurality of second solid pieces 43b are omitted. The configuration of the filter 40D except for this may be the same as the configuration of the filter 40C. The filter 40D also provides a high permeability for the target gas and a low permeability for the non-target substance.

The filter 40, the filter 40A, the filter 40B, the filter 40C, the filter 40D and the like can be applied to a gas sensor and the like.

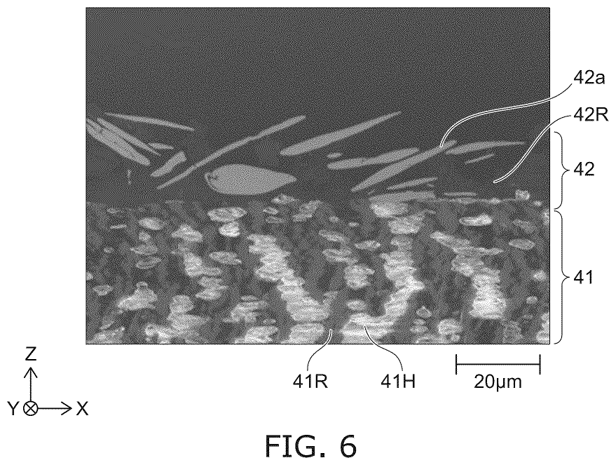

FIG. 6 is an electron micrograph image of the filter according to the first embodiment.

FIG. 6 is a SEM (Scanning Electron Microscope) image of a portion of the filter 40. As shown in FIG. 6, the plurality of holes 41H are provided. The second resin 42R of the second layer 42 is provided on a surface part of the plurality of holes 41H.

### SECOND EMBODIMENT

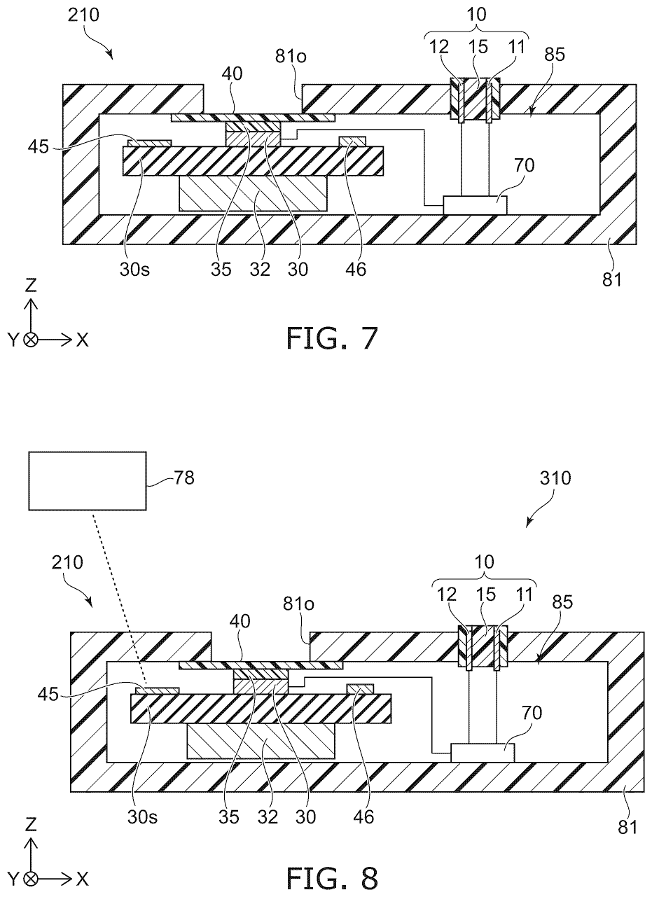

FIG. 7 is a schematic cross-sectional view illustrating a sensor according to the second embodiment.

As shown in FIG. 7, a sensor 210 (gas sensor) according to the embodiment includes a housing 81 and a detector 30. The detector 30 is provided in the housing 81. The detector 30 is configured to detect at least one selected from the group consisting of hydrogen, oxygen and VOC (Volatile Organic Compounds).

The housing 81 includes an opening 810 and a filter (for example, the filter 40) according to the first embodiment. By providing the filter 40, the target gas outside (out space) the housing 81 can efficiently pass through the filter 40. Entering of the non-target substance (water, etc.) existing outside (out space) the housing 81 into the housing 81 can be suppressed.

In the embodiment, the filter 40 may be in contact with the detector 30. Alternatively, the distance between the filter and the detector 30 is 1 cm or less. By providing the detector near the filter 40, the gas of detection target which has passed through the filter 40 can be detected with higher accuracy.

As shown in FIG. 7, the sensor 210 may include a second battery 32. The second battery 32 is configured to supply power to the detector 30. By providing the second battery 32, the detection target can be detected even in a place where commercial power is not supplied, for example.

In the sensor 210, the detector 30 is provided on a substrate 30s. A lid 35 may be provided between the detector and the filter 40. A humidity sensor 46 may be provided on the substrate 30s. The humidity in a space 85 inside the housing 81 may be monitored by the humidity sensor 46.

The sensor 210 may include a communicator 45. The communicator 45 is configured to transmit information related to a detection result of the detector 30 to an external device. The detection result includes, for example, information (data) on the concentration of the detection target. The transmission may be made, for example, by at least one of wire or wireless.

As shown in FIG. 7, the sensor 210 may include an electrochemical element 10. The electrochemical element 10 can, for example, at least one of dehumidify, humidify, generate ozone, generate oxygen, remove oxygen, or generate hydrogen. By applying a voltage between a first electrode 11 and a second electrode 12, at least one of dehumidification, humidification, ozone generation, oxygen generation, oxygen removal, or hydrogen generation can be performed. For example, dehumidification is performed by applying the voltage between the first electrode 11 and the second electrode 12. For example, water is absorbed by a first member 15 by the voltage application.

As shown in FIG. 7, the sensor 210 may include a controller 70. The controller 70 is configured to control at least one of the detector 30 and the electrochemical element 10.

### THIRD EMBODIMENT

FIG. 8 is a schematic cross-sectional view illustrating a sensor according to the third embodiment.

As shown in FIG. 8, a sensor system 310 according to the second embodiment includes the sensor 210 according to the second embodiment and a processing device 78. The processing device 78 may include, for example, a computer. The sensor 210 includes the communicator 45. The processing device 78 can process information based on a signal obtained from the communicator 45. The signal obtained from the communicator may include, for example, information (data) related to the detection result by the detector 30.

The processing of information (detection result) in the processing device 78 may include, for example, storing information (detection result). The processing of the information (detection result) may include, for example, comparing between the information (detection result) and a reference value. The processing device 78 may output an alert or the like according to the result of the comparing. The processing of the information (detection result) may include, for example, any calculation relating to the information (detection result). The calculation may include, for example, deriving the highest value or deriving the average value or the like.

Embodiments may include the following configurations (e.g., technical proposals).

### Configuration 1

A filter, comprising:
a first layer including a first resin, a plurality of holes being provided in the first resin, the first layer including a first face, at least a part of the plurality of holes reaching the first face; and
a second layer including a second resin, the second resin blocking at least a part of the plurality of holes reaching the first face.

### Configuration 2

The filter according to Configuration 1, wherein the first resin includes a fluorine compound.

### Configuration 3

The filter according to Configuration 2, wherein the second resin includes an acrylic resin.

### Configuration 4

The filter according to any one of Configurations 1 to 3, wherein a first thickness of the first layer is not less than 2 times and not more than 10,000 times a second thickness of the second layer.

### Configuration 5

The filter according to any one of Configurations 1 to 3, wherein a first thickness of the first layer is not less than 10 μm and not more than 5000 μm.

### Configuration 6

The filter according to any one of Configurations 1 to 3, wherein a second thickness of the second layer is not less than 0.1 μm and not more than 100 μm.

### Configuration 7

The filter according to any one of Configurations 1 to 6, wherein the second resin covers at least a part of the first face.

### Configuration 8

The filter according to any one of Configurations 1 to 7, wherein
the first face includes a first region in which the second resin is provided, and a second region in which the second resin is not provided, and
a ratio of an area of the first region to an area of the second region is not less than 0.01 and not more than 100.

### Configuration 9

The filter according to any one of Configurations 1 to 8, wherein the second layer further includes a plurality of first solid pieces.

### Configuration 10

The filter according to Configuration 9, wherein the plurality of first solid pieces include at least one of a metal, a metal oxide or a metal nitride.

### Configuration 11

The filter according to Configuration 9, wherein the plurality of first solid pieces include Fe, Cr and Ni.

### Configuration 12

The filter according to Configuration 9, wherein the plurality of first solid pieces include titanium oxide.

### Configuration 13

The filter according to any one of Configurations 1 to 12, further comprising a third layer including a third resin,
the first layer being located between the third layer and the second layer,
the first layer including a second face facing the third layer,
a part of the plurality of holes reaching the second face, and
the third resin blocking the part of the plurality of holes reaching the second face.

### Configuration 14

The filter according to Configuration 13, wherein the third resin includes an acrylic resin.

### Configuration 15

The filter according to Configuration 13 or 14, wherein a third thickness of the third layer is not less than 0.1 μm and not more than 100 μm.

### Configuration 16

The filter according to any one of Configurations 13 to 15, wherein the third resin covers at least a part of the second face.

### Configuration 17

The filter according to any one of Configurations 13 to 16, wherein
the second face includes a third region in which the third resin is provided, and a fourth region in which the third resin is not provided, and
a ratio of an area of the third region to an area of the fourth region is not less than 0.01 and not more than 100.

### Configuration 18

The filter according to any one Configurations 13 to 17, wherein the third layer further includes a plurality of second solid pieces.

### Configuration 19

A gas sensor, comprising:
a housing; and
a detector provided in the housing,
the housing including
an opening, and
the filter according to any one Configurations 1 to 18.

### Configuration 20

A gas system, comprising:
the gas sensor according to Configuration 19, and
the gas sensor including a communicator.

According to the embodiment, a filter, gas sensor, and gas sensor system capable of improving characteristics can be provided.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in in sensor system such as electrochemical elements, electrodes, first members, controllers, housings, detectors, first films, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all filters, gas sensors, and gas sensor systems practicable by an appropriate design modification by one skilled in the art based on the filters, gas sensors, and gas sensor systems described above as embodiments of the invention also are within the scope of the invention to the extent that the purport of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A filter, comprising:
a first layer including a first resin, a plurality of holes being provided in the first resin, the first layer including a first face, at least a part of the plurality of holes reaching the first face; and
a second layer including a second resin, the second resin blocking at least a part of the plurality of holes reaching the first face.

2. The filter according to claim 1, wherein the first resin includes a fluorine compound.

3. The filter according to claim 2, wherein the second resin includes an acrylic resin.

4. The filter according to claim 1, wherein a first thickness of the first layer is not less than 2 times and not more than 10,000 times a second thickness of the second layer.

5. The filter according to claim 1, wherein a first thickness of the first layer is not less than 10 μm and not more than 5000 μm.

6. The filter according to claim 1, wherein a second thickness of the second layer is not less than 0.1 μm and not more than 100 μm.

7. The filter according to claim 1, wherein the second resin covers at least a part of the first face.

8. The filter according to claim 1, wherein
the first face includes a first region in which the second resin is provided, and a second region in which the second resin is not provided, and
a ratio of an area of the first region to an area of the second region is not less than 0.01 and not more than 100.

9. The filter according to claim 1, wherein the second layer further includes a plurality of first solid pieces.

10. The filter according to claim 9, wherein the plurality of first solid pieces include at least one of a metal, a metal oxide or a metal nitride.

11. The filter according to claim 9, wherein the plurality of first solid pieces include Fe, Cr and Ni.

12. The filter according to claim 9, wherein the plurality of first solid pieces include titanium oxide.

13. The filter according to claim 1, further comprising a third layer including a third resin,
the first layer being located between the third layer and the second layer,
the first layer including a second face facing the third layer,
a part of the plurality of holes reaching the second face, and
the third resin blocking the part of the plurality of holes reaching the second face.

14. The filter according to claim 13, wherein the third resin includes an acrylic resin.

15. The filter according to claim 13, wherein a third thickness of the third layer is not less than 0.1 μm and not more than 100 μm.

16. The filter according to claim 13, wherein the third resin covers at least a part of the second face.

17. The filter according to claim 13, wherein
the second face includes a third region in which the third resin is provided, and a fourth region in which the third resin is not provided, and
a ratio of an area of the third region to an area of the fourth region is not less than 0.01 and not more than 100.

18. The filter according to claim 13, wherein the third layer further includes a plurality of second solid pieces.

19. A gas sensor, comprising:
a housing; and
a detector provided in the housing,
the housing including
an opening, and
the filter according to claim 1.

20. A gas system, comprising:
the gas sensor according to claim 19, and
the gas sensor including a communicator.
